(19) United States
(12) Patent Application Publication
(10) Pub. No.: US 2007/0059199 A1
(43) Pub. Date: Mar. 15, 2007

(54) Process for material treatment

(76) Inventor: Francois Jacques Labuschagne, Killarney Gardens (ZA)

Correspondence Address: STITES & HARBISON PLLC, 1199 NORTH FAIRFAX STREET, SUITE 900, ALEXANDRIA, VA, 22314, US

(21) Appl. No.: 10/557,937

(22) PCT Filed: May 18, 2004
(86) PCT No.: PCT/IB04/50738

§ 371 Date: Nov. 18, 2005

(30) Foreign Application Priority Data
May 19, 2003 (ZA) 2003/3841

(51) Int. Cl. A61L 2/08; B01J 49/00

(52) U.S. Cl. 422/26; 210/96.1

(57) ABSTRACT

The invention discloses a process for material treatment, which includes the step of subjecting used siliceous material to heat to obtain treated siliceous material. The process may be used to recycle used siliceous material. The invention also discloses a material treatment apparatus for treating used siliceous material, which includes heating means for heating used siliceous material to obtain treated siliceous material. The used siliceous material may be perlite and/or diatomaceous earth.

### FIELD OF INVENTION

The present invention relates to a process for material treatment. More particularly, the present invention relates to a process for treatment of siliceous materials.

### BACKGROUND TO INVENTION

Perlite, a naturally occurring siliceous volcanic glass, is an acid and glassy igneous rock which exhibits perlitic structure. The feature distinguishing perlite from other volcanic glasses is that when heated to a suitable point in its softening range, perlite expands from four to twenty times its original volume. This expansion process is due to the presence of two to six percent combined water in the crude perlite rock. When quickly heated to above 870 degrees Celsius, the crude rock pops in a manner similar to popcorn as the combined water vaporizes and creates countless tiny bubbles in the softened glassy particles. It is these tiny glass-sealed bubbles which are responsible for the amazing lightweight and other exceptional physical properties of expanded perlite.

The expansion process also creates one of perlite's most distinguishing characteristics: its white color. While the crude perlite rock may range from transparent to light gray to glossy black, the color of expanded perlite ranges from snowy white to grayish white. Expanded perlite can be manufactured to weigh from 32 kg/m³ to 240 kg/m³, making it adaptable for numerous uses, including filtration, horticultural applications, insulation, inert carriers and a multitude of filler applications. Since perlite is a form of natural glass, it is classified as chemically inert and has a pH of approximately 7.

Diatomaceous earth is a siliceous deposit occurring as a whitish powder consisting essentially of the frustules of diatoms. It is resistant to heat and chemical action, and thus used in fireproof cements, insulating materials, as an absorbent in the manufacture of explosives and as a filter. Diatoms are microscopic sized hard shelled creatures found in both marine and fresh waters. The diatom shells, covered in sharp spines, are dangerous to exoskeletal insects, but not to animals with internal skeletons. The spines of the diatom skeletons pierce the soft body tissues of insects between their hard exoskeletal plates and it is through these numerous microscopic wounds that the insect loses bodily moisture to the point of desiccating and dying. Creatures with internal skeletons such as humans, cattle and pets have means of resisting such damage and are therefore not harmed. Thus, by mixing a small amount of diatomaceous earth into stored grains and beans to deter insect infestations without having to remove the dust again before consuming them. Diatomaceous earth works in a purely physical and not chemical manner and thus has no chemical toxicity.

It is an object of the invention to suggest a novel process for material treatment.

### SUMMARY OF INVENTION

According to the invention, a process for material treatment, includes the step of subjecting used siliceous material to heat to obtain treated siliceous material.

The process may be used to recycle used siliceous material.

Also according to the invention, material treatment apparatus for treating used siliceous material include heating means for heating used siliceous material to obtain treated siliceous material.

Yet further according to the invention, siliceous material consists of a mixture of treated siliceous material and unused siliceous material.

The unused siliceous material may be unused refined siliceous material.

The used siliceous material may be substantially free of oil, wax, organic matter, residues from wine and fruit filtrations, and/or other filtration residues.

The used siliceous material may include about 10-30 weight % moisture.

The treated siliceous material may include about 0-4 weight % moisture.

The used siliceous material may be pre-washed at least once with water and/or steam prior to the step of subjection to heat.

The used siliceous material may include about 50-70 weight % wax and/or oil prior to the step of pre-washing.

The used siliceous material may have been used for constructional, industrial, filtration and/or horticultural applications.

The used siliceous material may be perlite and/or diatomaceous earth.

The used siliceous material may be moist.

Calcification and/or flash combustion of the siliceous material may take place.

The used siliceous material may be screw fed to the process and/or be suspended.

The step of heating may occur in a furnace dryer, CBR reactor, EBR reactor, fluidized bed reactor and/or other industrial dryers.

The reactor(s) and/or dryer(s) and/or accessories may be made of stainless steel.

The temperature of heating may be 600 to 850 degrees Celsius.

Heat may be provided with a flame, burner, electrodes, coils and/or electrically operated heating devices.

Means may be provided to prevent the siliceous material from contacting the flame.

The siliceous material inside the reactor and/or dryer may be fed by means of rotation and/or gravity and/or a screw feeder and/or a vibrating hopper.

Vapour may be released into the atmosphere.

The duration of treatment of the siliceous material may be about 1 to 5 seconds.

Chemicals may be used in the process.

The invention also extends to any product obtained by means of the aforesaid process.

### BRIEF DESCRIPTION OF ANNEXURES

The invention will now be described by way of example with reference to the accompanying annexures.

In the annexures there is shown in:

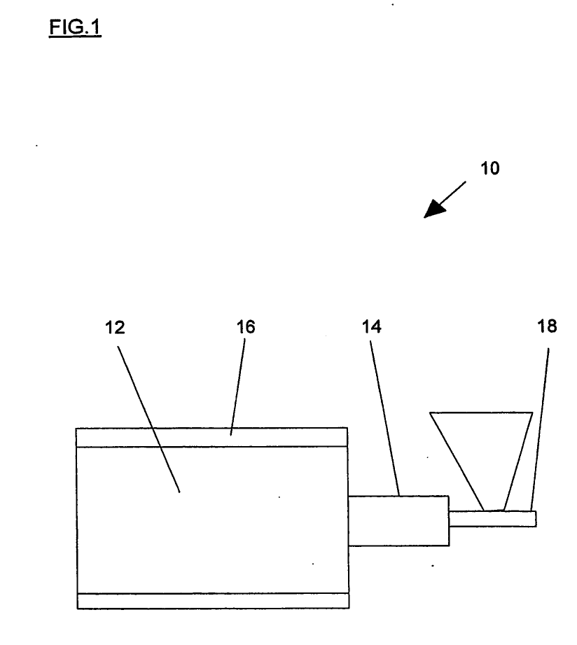

FIG. 1: a perspective view of a material treatment apparatus in accordance with the invention;

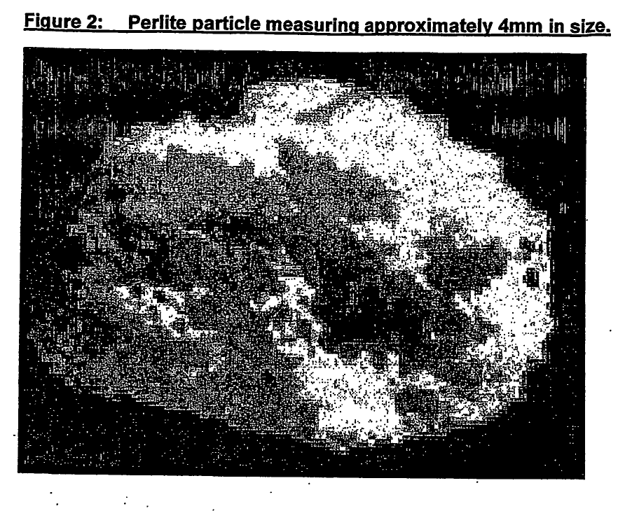

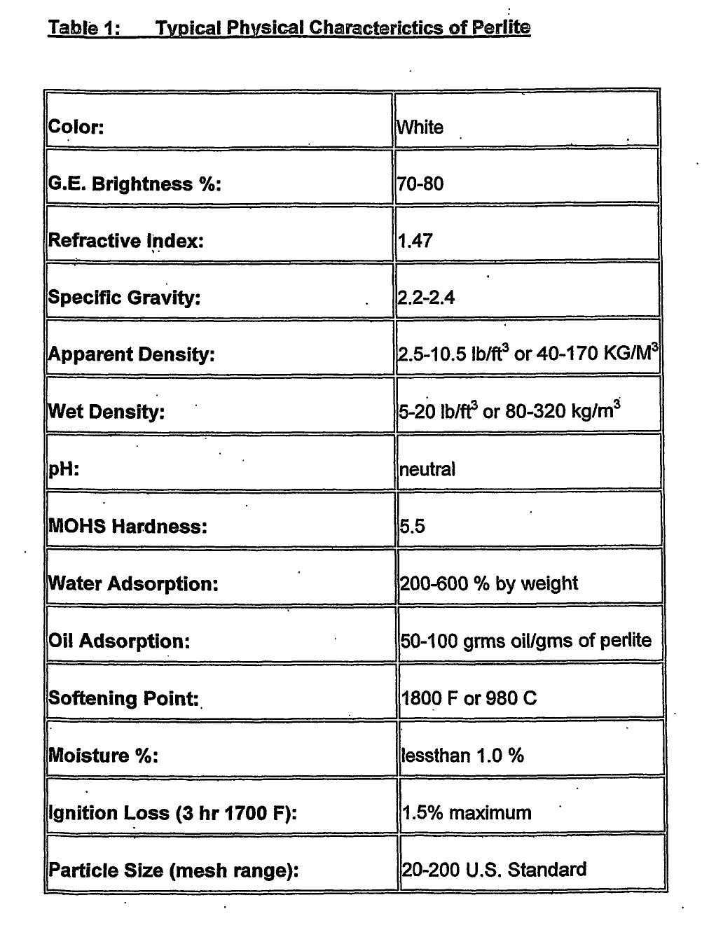

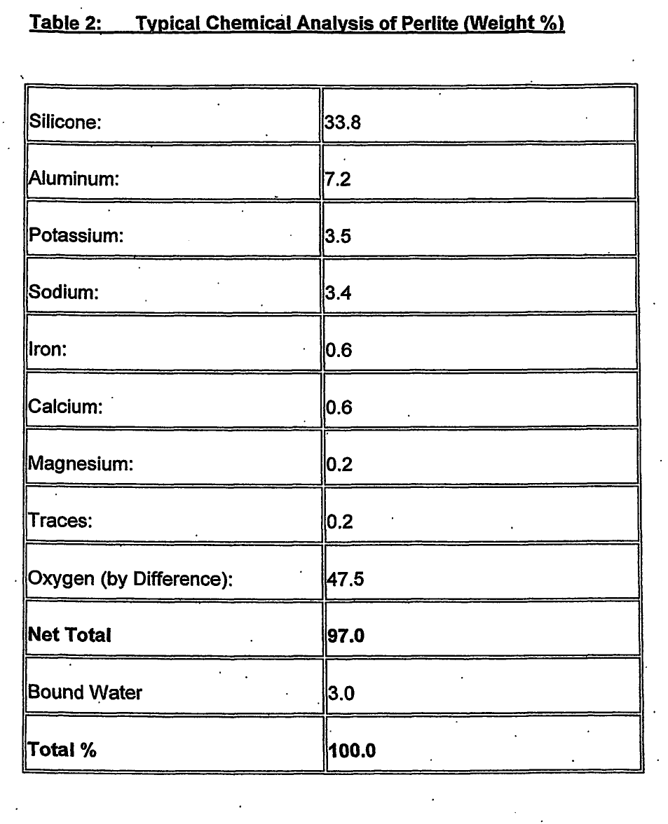

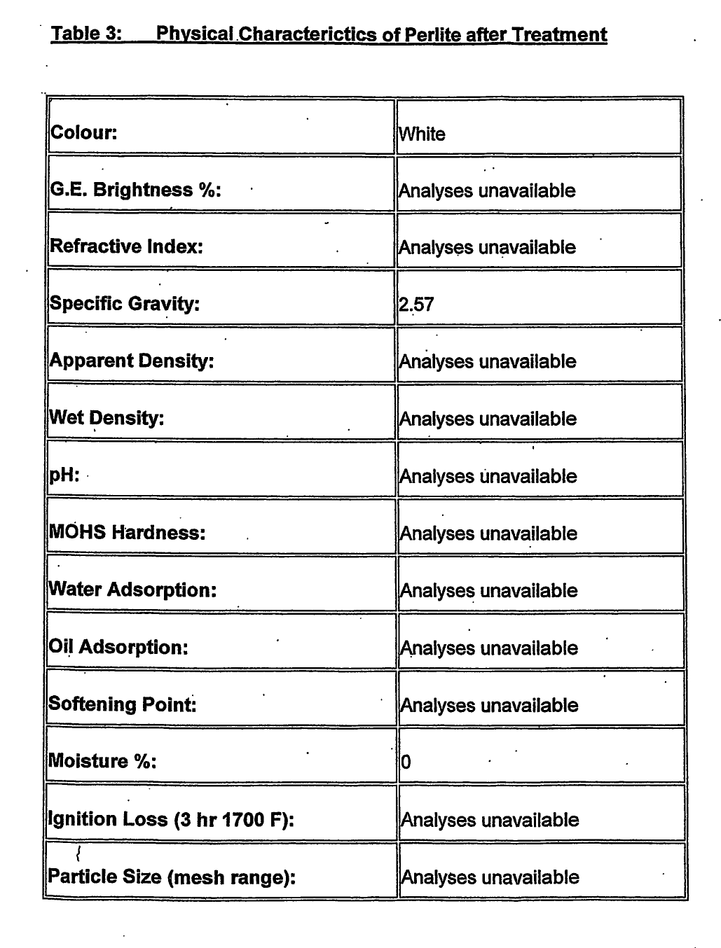

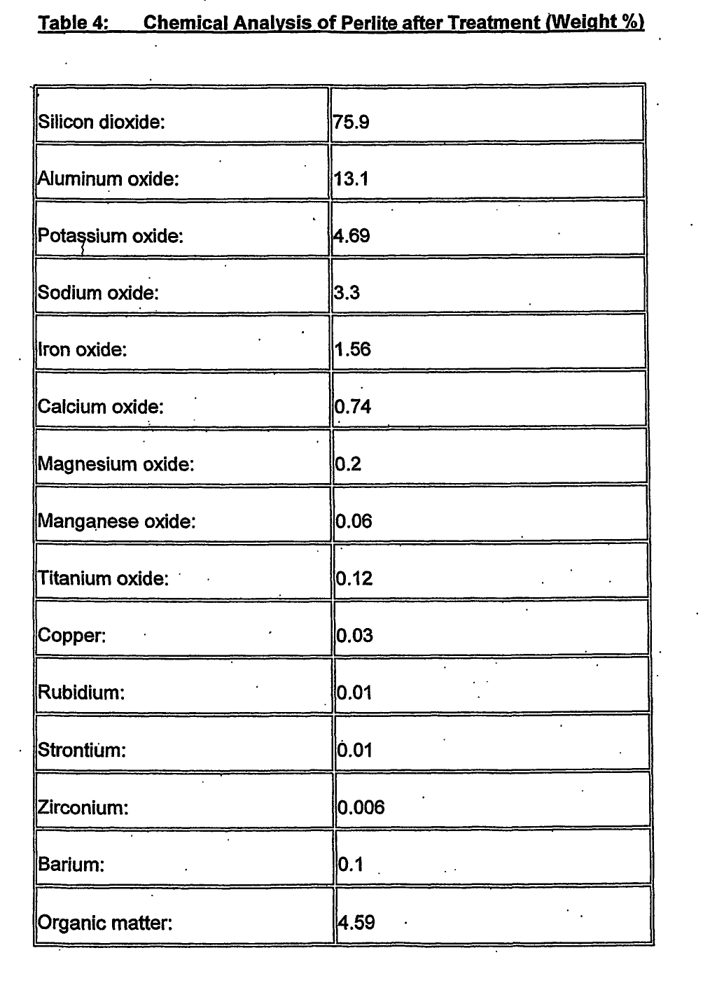

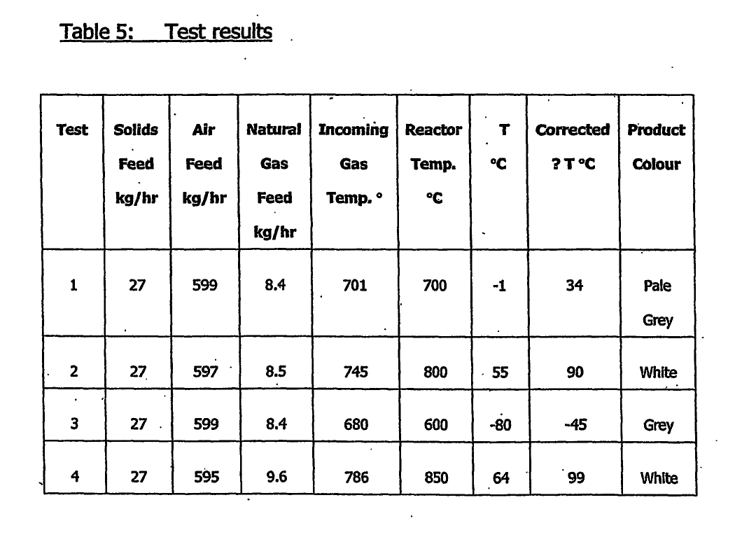

FIG. 2: a photograph of a perlite particle measuring approximately 4 mm in size;

Table 1: typical physical characteristics of raw perlite;

Table 2: typical chemical analysis of raw perlite;

Table 3: physical characteristics of treated perlite;

Table 4: chemical analysis of treated perlite; and

Table 5: test results of experiment.

### DETAILED DESCRIPTION OF ANNEXURES

Referring to FIG. 1, material treatment apparatus in accordance with the invention, generally indicated by reference numeral 10, is shown.

The material treatment apparatus 10 for treating used siliceous material includes a reactor/dryer 12, having a cylinder 14 and heating means (not shown). The reactor/dryer 12 is provided with an outer shell 16.

The material treatment apparatus 10 is used for recycling siliceous material by heating used siliceous material to obtain treated siliceous material. The used siliceous material is perlite or diatomaceous earth and is generally moist.

The used siliceous material is screw fed to the reactor/dryer 12 by means of a screw input 18. The reactor/dryer 12 is vertical. Normally a fluidized bed reactor is used in which the siliceous material is suspended. This facilitates the process of calcification or flash combustion of the siliceous material.

The maximum temperature of heating is normally 850 degrees Celsius and the heat is provided with a flame (not shown).

The siliceous material is suspended in the reactor/dryer 12 for a short period and then sucked out.

The siliceous material inside the cylinder 14 is fed by means of rotation and/or gravity and vapour is released into the atmosphere.

The duration of treatment of the siliceous material is about 1 to 5 seconds.

Chemical Composition and Mineralogy of Perlite

Water in raw (unexpanded) perlite occurs two main forms, free water and combined water. Free water is simply wetness on the surface of the rock. It does little to effect the expansion process except to make handling more difficult and to consume energy otherwise needed for expansion. It is the existance of combined water that gives perlite its ability to expand and become what some have termed “the most versatile mineral in the world”. The water has two effects: it lowers the softening point of the mineral, and it acts as the blowing agent which causes the molten rock to expand.

The presence of water is the result of a natural process. Perlite is found at the selvedge of lava flows, near the original surface, where the lava was able to chill quickly to form obsidian. In the subsequent years, the action of meteoric water permeating through the obsideian caused hydration to occur. The amount of water in the hydrated obsidian (perlite) can vary but is typically less than 4% in most commercial grades.

Experimental work seems to show that there are several different types of bond between the perlite and the combined water with varying amounts of water being released with different levels of energy.

Expansion of perlite requires very carefull delivery of heat and then removal of the particle from the heat zone. The particle must be heated quickly enough so that it becomes soft enough to expand before the water needed for expansion is driven off. This is most efficiently accomplished in specially designed furnaces which carry out the process in two or more stages and which include energy saving recuperation equipment.

Referring to FIG. 2, a photograph of a perlite particle measuring approximately 4 mm in diameter is shown.

Table 1 exhibits the typical physical characteristics of perlite prior to industrial use.

Table 2 exhibits the typical chemical characteristics of perlite prior to industrial use.

Table 3 exhibits the physical characteristics of perlite after treatment in accordance with the invention.

Table 4 exhibits the chemical characteristics of perlite after treatment in accordance with the invention.

The process for material treatment in accordance with the invention thus enables recycling of perlite and diatomaceous earth for re-use in similar or other applications.

Experiment

A waste stream consisting of a contaminated perlite which has been used as a filter medium in vegetable oil processing and in which most of the oils have been removed, is to be treated to remove the last small percentage of oil and to improve the appearance of the perlite to make it acceptable for reuse as a filter aid. Test results showed that the perlite could be cleaned with three passes at about 700° C. in a pilot EBR unit. It is important to note that the particle size distribution of the feed varies from very fine up to about 150μ. A Sample of about 85 kg of contaminated perlite was used for these tests. The composition of the stream consisted of about 66% perlite, 30% moisture and 4% organics.

A large feeder with an open flight screw was used, with only occasional bridging of the material. A flat bar clamped to the vibrating hopper of the feeder kept the material just above the screw in motion and allowed the tests to proceed. The resultant minimum feed rate that could be set was 27 kg/hr and was used for all tests.

A series of four tests were performed at temperatures of 600, 700, 800 and 850° C. using a solids feed rate of 27 kg/hr and in the same way. The reactor was brought up close to the temperature for the test, the feed was started and the inlet air temperature was adjusted to compensate for any net exotherm or net endotherm caused by the introduction of the solid feed and to bring the reactor temperature back to the test condition. The reactor was run for about 20 minutes to ensure steady state operation. A sample of the feed was then collected.

Once the reactor had been brought up to the required temperature, a 35° C. drop in temperature was observed from the inlet gas measured temperature to the temperature in the centre of the reactor. This is normal in the pilot plant where much higher than normal heat losses occur. This temperature difference was used to correct the measured temperature (T) for each test (see Table 5).

Though the feeding of the material was tricky, once in the reactor the material processed extremely well. All tests yielded free flowing powder products, of varying colours as stated in Table 5. The results of the pilot plant are indicative of a fairly strong exothermic process particularly at 800° C. and above. The endotherm and the grey colour of the ash processed at 600° C. suggest that combustion of the fats and waxes is incomplete at this temperature resulting in the creation of carbon which colours the perlite product.

A theoretical heat balance undertaken for an operating temperature of 800° C. suggests that the temperature (T) should only be about 13° C. Given the higher heat losses from the pilot plant still registering a 90° C. temperature (T), this would suggest that the organic content of the sample tested was higher, or the moisture content lower than the theoretical composition provided.

Use

Due to the outstanding insulating characteristics and light weight, perlite is widely used as a loose-fill insulation in masonry construction. In this application, free-flowing perlite loose-fill masonry insulation is poured into the cavities of concrete block where it completely fills all cores, crevices, mortar areas and ear holes. In addition to providing thermal insulation, perlite enhances fire ratings, reduces noise transmission and it is rot, vermin and termite resistant perlite is also ideal for insulating low temperature and cryogenic vessels.

When perlite is used as an aggregate in concrete, a lightweight, fire resistant, insulating concrete is produced that is ideal for roof decks and other applications. Perlite can also be used as an aggregate in Portland cement and gypsum plasters for exterior applications and for the fire protection of beams and columns. Other construction applications include under-floor insulation, chimney linings, paint texturing, gypsum boards, ceiling tiles and roof insulation boards.

Industrial applications for perlite are the most diverse, ranging from high performance fillers for plastics to cements for petroleum, water and geothermal wells. Other applications include its use as a filter media for pharmaceuticals, food products, chemicals and water for municipal systems and swimming pools. Additional applications include its use as an abrasive in soaps, cleaners and polishes and a variety of foundry applications utilizing perlite's insulating properties and high heat resistance. This same heat resistant property is taken advantage of when perlite is used in the manufacture of refractory bricks, mortars, and pipe insulation.

In horticultural applications, perlite is used throughout the world as a component of soilless growing mixes where it provides aeration and optimum moisture retention for superior plant growth. For rooting cuttings, 100% perlite is used.

Other benefits of horticultural perlite are its neutral pH and the fact that it is sterile and weed-free. In addition, its light weight makes it ideal for use in container growing.

Other horticultural applications for perlite are as a carrier for fertilizer, herbicides and pesticides and for pelletizing seed. Horticultural perlite is as useful to the home gardener as it is to the commercial grower. It is used with equal success in greenhouse growing, landscaping applications and in the home in house plants.

### LIST OF REFERENCE NUMERALS

10 Material treatment apparatus
12 Reactor/dryer
14 Cylinder
16 Outer shell
18 Screw input

1-22. (canceled)

23. A process for material treatment for recycling used siliceous material, which includes the step of heating by means at least one process selected from the group consisting of calcification and flash combustion, the used siliceous material being selected from the group comprising perlite and diatomaceous earth and having been used in at least one of the applications selected from the group consisting of constructional, industrial, filtration and horticultural applications.

24. A process as claimed in claim 23, in which the used siliceous material is substantially free of at least one component selected from the group consisting of oil, wax, organic matter, residues from wine, residues from fruit filtrations, and other filtration residues.

25. A process as claimed in claim 23, in which the used siliceous material includes about 10-30 weight % moisture.

26. A process as claimed in claim 23, in which the treated siliceous material includes about 0-4 weight % moisture.

27. A process as claimed in claim 23, in which prior to the step of subjection to heat, the used siliceous material is pre-washed at least once with water.

28. A process as claimed in claim 23, in which prior to the step of subjection to heat, the used siliceous material is pre-washed at least once with steam.

29. A process as claimed in claim 23, in which prior to the step of pre-washing the used siliceous material includes 50-70 weight % of wax.

30. A process as claimed in claim 23, in which prior to the step of pre-washing the used siliceous material includes 50-70 weight % of oil.

31. A process as claimed in claim 23, in which the used siliceous material is screw fed to the process.

32. A process as claimed in claim 23, in which the used siliceous material is suspended.

33. An process as claimed in claim 23, in which the step of heating occurs in at least one apparatus selected from the group consisting of a furnace dryer, a CBR reactor, an EBR reactor, and an industrial dryer.

34. An process as claimed in claim 23, in which the temperature of heating is between 600 and 850 degrees Celsius.

35. A material treatment apparatus for treating used siliceous material for recycling used siliceous material, which includes at least one heating means selected from the group consisting of calcification and flash combustion and is adapted to be used for heating used siliceous material to obtain treated siliceous material, the used siliceous material being selected from the group consisting of perlite and diatomaceous earth and which has been used in at least one of the applications selected from the group comprising constructional, industrial, filtration and horticultural applications.

36. An apparatus as claimed in claim 35, which includes at least one component selected from the group consisting of a furnace dryer, a CBR reactor, an EBR reactor, a fluidized bed reactor and an industrial dryer.

37. An apparatus as claimed in claim 35, in which the siliceous material is suspended.

38. An apparatus as claimed in claim 35, in which the siliceous material is screw-fed.

39. An apparatus as claimed in claim 35, in which the heating means is made of stainless steel

40. An apparatus as claimed in claim 35, in which the heating means includes at least one device selected from the group consisting of a flame, a burner, an electrode, a coil, an electrically operated heating device and gas operated heating devices.

41. A apparatus as claims in claim 35, in which the siliceous material inside the cylinder are fed by means of at least one device selected from the group consisting of a rotation feeder, a gravity feeder, a screw feeder and a vibrating hopper.

42. An apparatus as claimed in claim 35, which is adapted to release vapor to the atmosphere.

43. An apparatus as claimed in claim 35, in which the temperature of heating is between 600 to 850 degrees Celsius.

44. An apparatus as claimed in claim 35, in which the duration of treatment of the siliceous material is about 1 to 5 seconds.

45. A siliceous product, which is obtained by means of the process as claimed in claim 23.
